(19) United States
(12) Patent Application Publication
(10) Pub. No.: US 2025/0107858 A1
(43) Pub. Date: Apr. 3, 2025

(54) SYSTEM AND METHOD FOR ENGAGING AN INSTRUMENT OF A COMPUTER-ASSISTED SYSTEM

(71) Applicant: Intuitive Surgical Operations, Inc., Sunnyvale, CA (US)

(72) Inventors: Gabriel F. Brisson, Sunnyvale, CA (US); Gregory W. Dachs, II, San Mateo, CA (US)

(73) Assignee: Intuitive Surgical Operations, Inc., Sunnyvale, CA (US)

(21) Appl. No.: 18/903,188

(22) Filed: Oct. 1, 2024

Related U.S. Application Data
(60) Provisional application No. 63/542,003, filed on Oct. 2, 2023.

(51) Int. Cl. A61B 34/30; A61B 34/00; A61B 34/20

(52) U.S. Cl. CPC A61B 34/30; A61B 2034/2046; A61B 2034/715

(57) ABSTRACT

A computer-assisted system includes an instrument. The instrument includes a receive interface configured to couple with a drive interface, the receive interface including a first receive coupling with a set of first receive features disposed on the first receive coupling, each first receive feature in the set physically configured for mechanically engaging with a same first drive feature disposed on a first drive coupling of the drive interface, and a transmission element coupled with the first receive coupling such that mechanical energy transmitted by the first drive coupling to the first receive coupling is transmitted to the transmission element.

### CROSS-REFERENCE TO RELATED APPLICATIONS

This application claims priority to and the benefit of the filing date of U.S. Provisional Patent Application 63/542,003, filed on Oct. 2, 2023, which is hereby incorporated by reference herein in its entirety.

### BACKGROUND

### Field of Invention

The present invention generally provides improved computer-assisted devices, systems, and methods.

### Overview

Computer-assisted systems can be used to perform a task at a worksite. For example, a computer-assisted system may comprise a handheld, motorized tool assembly. As another example, a computer-assisted system may comprise a robotic system, and may include one or more robotic manipulators to manipulate instruments for performing the task.

Example computer-assisted systems include industrial and recreational robotic systems. Example computer-assisted systems also include medical robotic systems used in procedures for diagnosis, non-surgical treatment, surgical treatment, etc.

Some computer-assisted systems include one or more instruments that are articulated in order to perform various procedures. The computer-assisted system can be automated, semi-automated, teleoperated, etc. In a teleoperated example, a human operator manipulates one or more leader input controls to command motion of one or more follower instruments located in a workspace. In some examples, the teleoperated system is configured to support an instrument that includes an imaging device, such as an endoscope or a camera, that enables the operator to observe the workspace. In some instances, the field of view of the imaging device is directed to enable the operator to see the instrument(s) as the operator commands motion of the instrument(s).

Different types of instruments may be interchangeably used. Accordingly, with some computer-assisted systems, instruments may be coupled to, removed from, and/or re-coupled to the computer-assisted system. A more efficient, more reliable, and/or easier-to-perform engagement of instruments to computer-assisted systems is, therefore, highly desirable.

### SUMMARY

In general, in one aspect, one or more embodiments relate to a computer-assisted system comprising: an instrument comprising: a receive interface configured to couple with a drive interface, the receive interface comprising a first receive coupling with a plurality of first receive features disposed on the first receive coupling, each first receive feature of the plurality of first receive features physically configured for mechanically engaging with a same first drive feature disposed on a first drive coupling of the drive interface, and a transmission element coupled with the first receive coupling such that mechanical energy transmitted by the first drive coupling to the first receive coupling is transmitted to the transmission element.

In general, in one aspect, one or more embodiments relate to a method for engaging an instrument of a computer-assisted system, comprising: detecting, with a control system of the computer-assisted system, a physical coupling of a receive interface of the instrument with a drive interface of the computer-assisted system, wherein the receive interface comprises a receive coupling and the drive interface comprises a drive coupling; causing, with the control system and in response to detecting the physical coupling, motion of the drive coupling to engage the drive coupling with the receive coupling, wherein the receive coupling has a plurality of engagement options with the drive coupling; and confirming, with the control system, whether the drive coupling has engaged with the receive coupling.

Other aspects of the invention will be apparent from the following description and the appended claims.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows an example computer-assisted system, in accordance with one or more embodiments.

FIG. 2 shows an example of a manipulator assembly, in accordance with one or more embodiments.

FIG. 3 shows an example instrument, in accordance with one or more embodiments.

FIGS. 4A and 4B show combination elements of a robotic arm and an instrument, in accordance with one or more embodiments.

FIGS. 5A-5H show example couplings that can be drive couplings or receive couplings, in accordance with one or more embodiments.

FIG. 6 shows a flowchart describing an example method for engaging an instrument, in accordance with one or more embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements, and is not to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

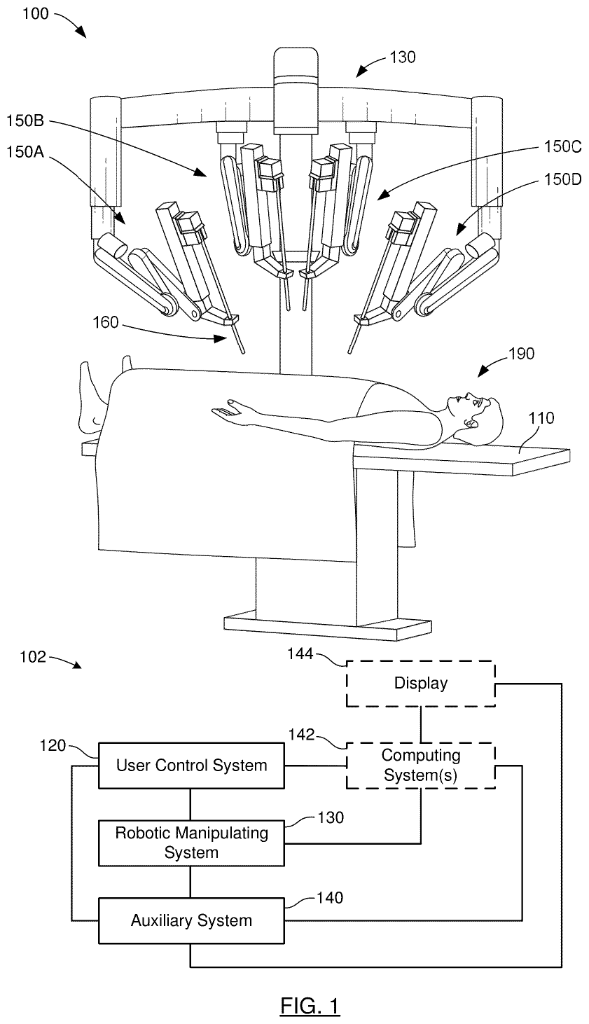

Referring now to the drawings, in which like reference numerals represent like parts throughout the several views, FIG. 1 shows an example computer-assisted system (100), in accordance with one or more embodiments.

While FIG. 1 shows the computer-assisted system (100) as a medical system, the following description is applicable to other scenarios and systems, e.g., medical scenarios or systems that are non-surgical, non-medical scenarios or computer-assisted systems, etc.

In the example, a diagnostic or therapeutic medical procedure is performed on a patient (190) on an operating table (110). The computer-assisted system (100) may include a robotic manipulating system (130) (e.g., a patient-side robotic device in a medical example). The robotic manipulating system (130) may include at least one robotic arm (150A, 150B, 150C, 150D), each of which may support a removably coupled instrument (160) (also called tool (160)). In the illustrated procedure, the instrument (160) may enter the workspace through an entry location (e.g., enter the body of the patient (190) through a natural orifice such as the throat or anus, or through an incision), while an operator (not shown) views the worksite (e.g., a surgical site in the surgical scenario) through a user interface system (120).

An image of the worksite may be obtained by an imaging instrument (160) comprising an imaging device (e.g., an endoscope, an optical camera, an ultrasonic probe, etc. in a medical example). The imaging instrument (160) can be used for imaging the worksite, and may be manipulated by one of the robotic arms (150A, 150B, 150C, 150D) of the robotic manipulating system (130) so as to position and orient the imaging instrument. The auxiliary system (140) may process the captured images in a variety of ways prior to any subsequent display. For example, the auxiliary system (140) may overlay the captured images with a virtual control interface prior to displaying the combined images to the operator via the user interface system (120) or other display systems located locally or remotely from the procedure. One or more separate displays (144) may also be coupled with a control system (142) and/or the auxiliary system (140) for local and/or remote display of images, such as images of the procedure site, or other related images.

The number of instruments (160) used at one time generally depends on the task and space constraints, among other factors. If it is appropriate to change, clean, inspect, or reload one or more of the instruments (160) being used during a procedure, an assistant (not shown) may remove the instrument (160) from the robotic arm (150A, 150B, 150C, 150D), and replace it with the same instrument (160) or another instrument (160).

The computer-assisted system (100) may include a control system (142). The control system (142) may be used to process input provided by the user interface system (120) from an operator, such as to control the computer-assisted system (100). The control system (142) may also be used to process signals from other devices, from sensors, from any networks to which the control system (142) connects, etc. Example sensors include those associated with actuators or joints of the computer-assisted system, such as motor encoders, rotary or linear joint encoders, torque sensors, current sensors, accelerometers, force sensors, inertial measurement units, optical or ultrasonic sensors or imagers, RF sensors, etc. The control system may further be used to provide an output, e.g., a video image for display by the display (144). The control system (142) may further be used to control the robotic manipulating system (130).

The control system (142) may include one or more computer processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities.

A computer processor of the control system (142) may be part or all of an integrated circuit for processing instructions. For example, the computer processor may be one or more cores or micro-cores of a processor. The control system (142) may also communicate with one or more input devices, such as a touchscreen, keyboard, mouse, microphone, touchpad, electronic pen, or any other type of input device.

A communication interface of the control system (142) may include an integrated circuit for connecting the control system (142) to a network (not shown) (e.g., a local area network (LAN), a wide area network (WAN) such as the Internet, mobile network, or any other type of network) and/or to another device, such as another control system (142).

Further, the control system (142) may communicate with one or more output devices, such as a display device (e.g., a liquid crystal display (LCD), a plasma display, touchscreen, organic LED display (OLED), projector, or other display device), a printer, a speaker, external storage, or any other output device. One or more of the output devices may be the same or different from the input device(s). Many different types of control systems exist, and the aforementioned input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the invention.

A control system (142) may be connected to or be a part of a network. The network may include multiple nodes. Each node may correspond to a computing system, or a group of nodes. By way of an example, embodiments of the disclosure may be implemented on a node of a distributed system that is connected to other nodes. By way of another example, embodiments of the invention may be implemented on a distributed computing system having multiple nodes, where each portion of the disclosure may be located on a different node within the distributed computing system. Further, one or more elements of the aforementioned computing system may be located at a remote location and connected to the other elements over a network.

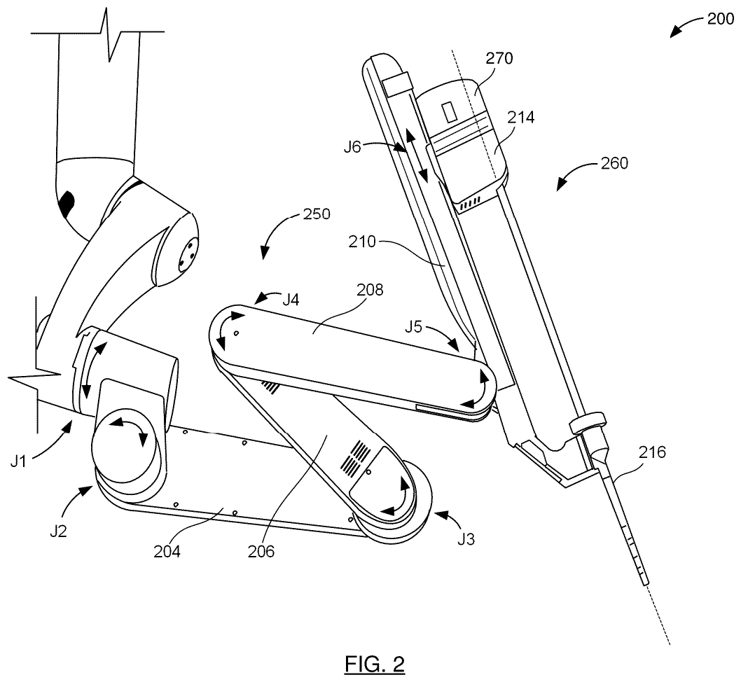

An example of a manipulator assembly (200) in accordance with embodiments of the present disclosure is shown in FIG. 2. A manipulator assembly (200) may include a robotic arm (250) and an instrument (260). The robotic arm (250) may correspond to the robotic arm (150A, 150B, 150C, 150D) in FIG. 1. As described above, during operation, the robotic arm (250) generally supports an instrument (260) extending distally from the robotic arm (250), and effects movements of the instrument (260).

In minimally invasive scenarios, an instrument (260) may be positioned and manipulated through an entry location, so that a kinematic remote center is maintained at the entry location. Images of the worksite, taken by an imaging device of an imaging instrument such as an optical camera, may include images of the distal ends of the instruments (260) when the instruments (260) are positioned within the field-of-view of an imaging device.

In the example shown, a distal instrument holder (214) facilitates removal and replacement of the mounted instrument.

As may be understood with reference to FIG. 1, robotic arms (250) are proximally mounted to a base of the robotic assembly. Alternatively, robotic arms (250) may be mounted to separate bases that may be independently movable, e.g., by the robotic arms (250) being mounted to single-robotic-arm carts, being provided with mounting clamps that allow mounting of the robotic arms (250) directly or indirectly to the operating table at various locations, etc. An example robotic arm (250) includes a plurality of links and joints extending between the proximal base and the distal instrument holder.

In embodiments such as shown for example in FIG. 2, a robotic arm includes multiple joints (such as revolute joints J1, J2, J3, J4, and J5, and prismatic joint J6) and links (204, 206, 208, and 210). The joints of the robotic arm, in combination, may or may not have redundant degrees of freedom.

In the example shown, the instrument (260) may be releasably mounted on an instrument holder (214). The instrument holder (214) may translate along a linear guide formed by the prismatic joint (J6) at the link (210). Thus, the instrument holder (214) may provide in/out movement of the instrument (260) along an insertion axis. The link (210) may further support a cannula (216) through which the instrument shaft of the instrument (260) extends. Alternatively, the cannula (216) may be mechanically supported by another component of the robotic arm (250) or may not be supported at all.

Actuation of the instrument (260) in one or more degrees may be provided by actuators of the manipulator assembly. These actuators may be integrated in the instrument holder (214), or drive drivetrains in the instrument holder, and may actuate the instrument (260) via the transmission assembly (270).

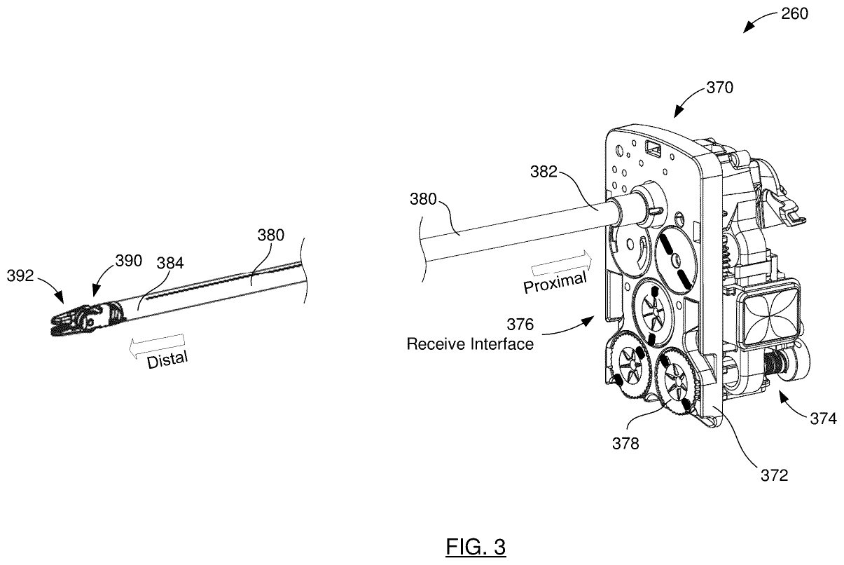

While FIG. 2 shows an external housing of the transmission assembly (270), additional components of the transmission assembly (270) are discussed below. Similarly, additional components of the instrument holder (214) are discussed below, including a detailed description of an example interfacing of the instrument (260) with the robotic arm (250) at the instrument holder (214) and the transmission assembly (270). Also, a more detailed description of the example instrument (260) is provided below in reference to FIG. 3.

Referring to FIG. 3, an example instrument (260) includes a transmission assembly (370), an instrument shaft (380), a wrist assembly (390), and an end effector (350) at a distal portion (384) of the instrument shaft (380).

The instrument shaft (380), in one or more embodiments, has a proximal portion (382) terminating at a chassis (372) of the transmission assembly (370) and a distal portion (384) terminating at the wrist assembly (390) of the instrument (260). The instrument shaft (380) may be any suitable shaft that couples the wrist assembly (390) and the end effector (392) to the transmission assembly (370). In one or more embodiments, the shaft (380) defines at least one passageway through which one or more tensions members (e.g., cables, metal bands, not shown) or other transmission members suitable for the transmission of mechanical energy such as movement, force, or torque may be routed from the transmission assembly (370) towards the wrist assembly (390). In some embodiments, other components (e.g., electrical wires, push-rods, optical fibers, etc.) may also be routed through the shaft.

The wrist assembly (390) is configured to provide a rotational degree of freedom (e.g., a pitch rotation) of the end effector (392). The end effector (392) is configured with a yaw rotation degree of freedom that may further be used for opening and closing jaw elements of the end effector (392). Depending on the design, one, two, or more tension members may be used to control a degree of freedom for the instrument. In an example with a jawed instrument, three pairs of tension members are used in end effector control (six tension members total), with a first pair of tension members used to control end effector pitch (rotation about a first end effector axis), a second pair of tension members used to control end effector yaw (rotation about a second end effector axis perpendicular to the first end effector axis), and a third pair of tension members used to control jaw opening. In another example with a jawed instrument, three pairs of tension members are also used in end effector control, with a first pair of tension members used to control end effector pitch (rotation about a first end effector axis), a second pair of tension members used to control rotational motion of a first finger of the jaw in a “yaw” direction (rotation about a second end effector axis perpendicular to the first end effector axis), and a third part of tension members used to control rotational motion of a second finger of the jaw in the “yaw” direction (rotation about the second end effector axis). In this example, coordinated motion of the two fingers of the jaw provides end effector yaw or opening-closing motion of jaws. In another example, some of the tension members are coupled to multiple degrees of freedom, and four tension members are used in combination to control a pitching, a yawing, and a jaw opening for a jawed instrument.

In one or more embodiments, the tensions members are routed through the shaft to other elements of the transmission assembly (370) and may be operated by transmission elements (374). In the example of FIG. 3, the transmission elements (374) include capstans. In one example with four tension members, a separate capstan (or more generally, transmission element (374)) may be provided for each of the tension members. Four capstans may, thus, be provided to actuate the wrist assembly (390) and the end effector (392), in the example. Operation of the four capstans causes movement of the four tension members to produce the desired movement (pitch, yaw, and/or grip) of the end effector (392). Specifically, a first capstan may move a first tension member in a proximal direction (pulling the tension member) by winding up the first tension member. Simultaneously, a second capstan may move a second tension member in a distal direction (releasing the tension member) by unwinding the second tension member when the first and the second tension members operate in an antagonistic manner. The second tension member may also be operated independently from the first tension member.

In the example of FIG. 3, the transmission elements (374) further include gears (e.g., spur gears) for rolling or rotation of the instrument shaft (380), the wrist assembly (390), and the end effector (392) about a roll axis of the instrument shaft (380). Accordingly, in the example of FIG. 3, the transmission assembly (370) includes example five transmission elements (374) that are capstan and tension member-based, or gear-based. Other types of transmission elements such as, for example, metal bands, rubber belts, metal wires, pulleys, screw drives, chains and sprockets, etc., may be used without departing from the disclosure.

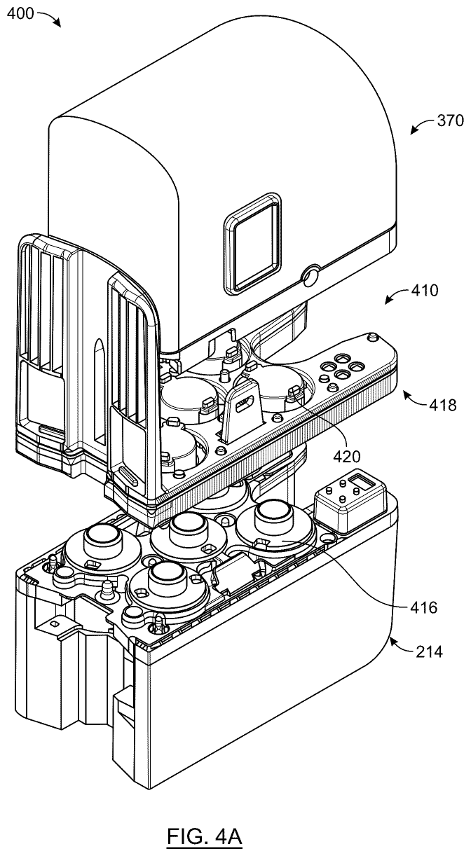

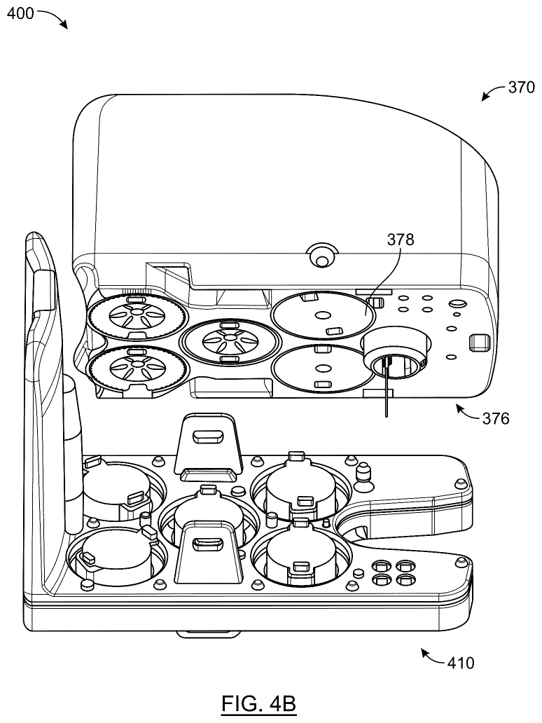

In one or more embodiments, the transmission assembly (370) further includes receive couplings (378). In the example of FIG. 3, one receive coupling (378) may be coupled with one of the transmission elements (374). Accordingly, the transmission assembly (370), in the example of FIG. 3, includes five receive couplings (378). Each receive coupling (378) may be designed to engage with a corresponding drive coupling (discussed in reference to FIGS. 4A and 4B) of the robotic arm (250) or other repositionable structure. When a receive coupling (378) is engaged with a drive coupling, mechanical energy may be transmitted over the combination of the drive coupling and a receive coupling. For example, a torque or velocity may be transmitted from an electric motor or any other type of actuator to a capstan.

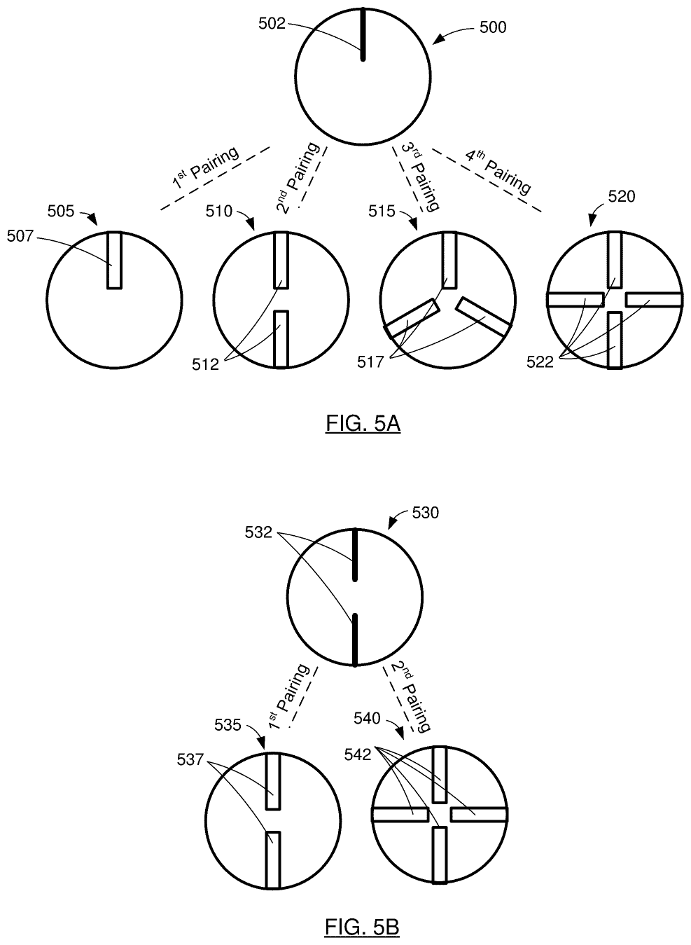

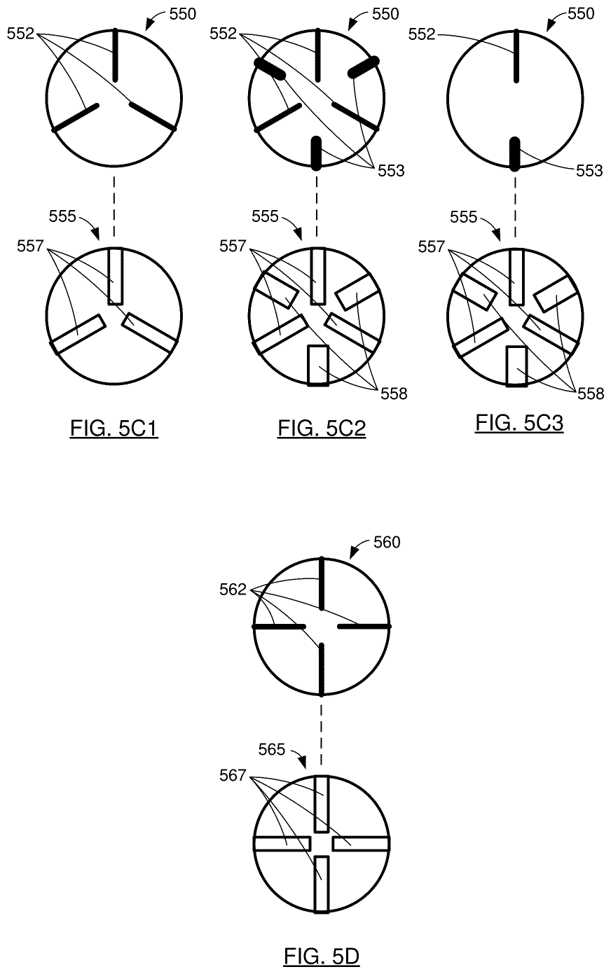

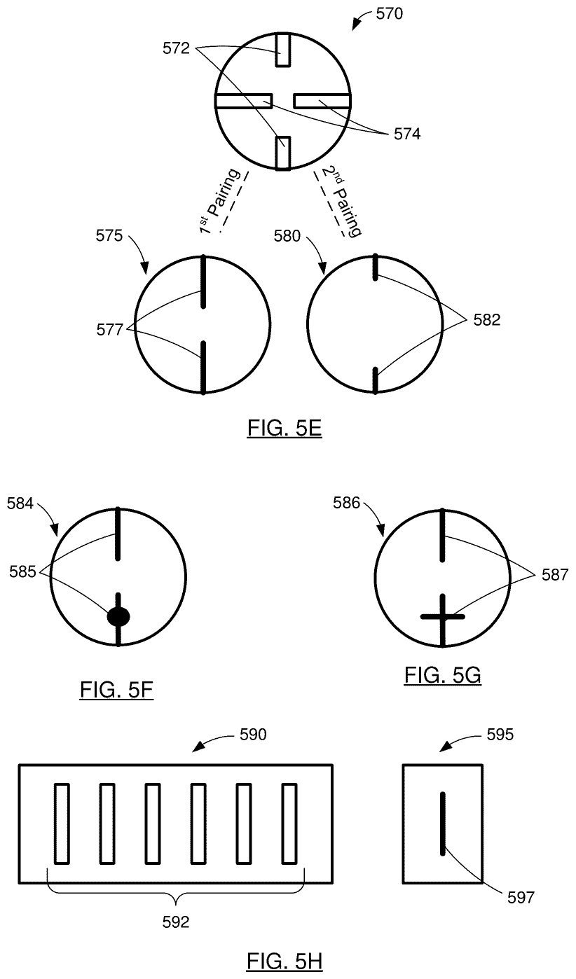

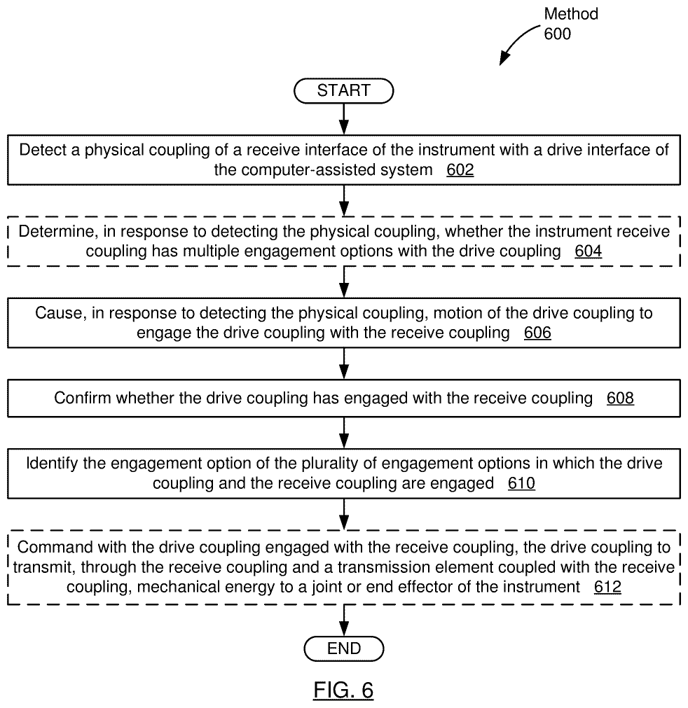

A detailed description of receive couplings and drive couplings, including their geometries and their engagement, is provided below in reference to FIGS. 5A-5H. Further operations involved in engaging a receive coupling and a corresponding drive coupling are described below in reference to the flowchart of FIG. 6.

The chassis (372) may provide structural support for mounting and aligning the components (e.g., the transmission elements and the receive couplings) of the transmission assembly (370). The chassis (372) may further function as part of a housing of the transmission assembly (370).

In one or more embodiments, the chassis (372) may also include external features (not shown) that interface with elements of a drive interface, which may be part of the instrument holder (214) to releasably lock the instrument (260) to the instrument holder (214) or other repositionable structure. Recesses, clips, or other elements may be included in the external features. Additional details are provided below in reference to FIGS. 4A and 4B.

The receive interface (376) comprises the combination of components and features of the transmission assembly (370) that, when the instrument is mounted to the robotic arm (250), contacts the robotic arm (250) or an intervening adapter mounted to the robotic arm (250). The drive interface comprises the combination of components and features of the robotic arm (250) or other repositionable structure that, when the instrument is mounted to the robotic arm (250), contact the instrument or an intervening adapter mounted to the instrument. In reference to FIGS. 4A and 4B, elements of the receive interface (376) may include, for example, the previously described external features (recesses, clips, etc.) for releasably locking the instrument (260) to the robotic arm (250), and/or the previously described receive couplings (378).

FIG. 4A shows an exploded view of an example of combination elements (400) of a robotic arm and an instrument, in accordance with one or more embodiments. FIG. 4B provides a detail view of additional elements of the combination elements (400) of the robotic arm and the instrument.

In one or more embodiments, instrument holder drive couplings (416) are actuated by actuators (not shown). These actuators may be located in the instrument holder (214). The instrument holder drive couplings (416), when engaged with the receive couplings (378), either directly or indirectly, may be used to actuate the instrument (260) in its degrees of freedom. The instrument holder drive couplings (416) on the instrument holder (214) may directly mechanically engage with the receive couplings (378) on the transmission assembly (370) of the instrument (260), without any intervening components. Alternatively, the engaging of the instrument holder drive couplings (416) with the receive couplings (378) may be indirect. In the example shown in FIGS. 4A and 4B, one intervening adapter (410) provides a barrier between the instrument holder (214) and the transmission assembly (370). Other embodiments may utilize two or more intervening adapters.

In one embodiment, the intervening adapter (410) is disposed on the instrument holder (214), thereby establishing a separation between the instrument holder (214) and the transmission assembly (370). In applications that benefit from a cleaner environment, the intervening adapter (410) may be part of a larger assembly that separates the instrument space from the robotic arm space. For example, in medical applications or other applications that require a sterile environment, the intervening adapter (410) may be an instrument sterile adapter portion of a sterile drape. In such a configuration, the intervening adapter (410) helps to establish a barrier between the less-clean (e.g., non-sterile) components (e.g., the robotic arm (250)) and the clean (e.g., sterile) components (e.g., the instrument (260)). In various embodiments, the intervening adapter (410) between the instrument (260) and the robotic arm (250) provides a coupling region for the instrument (260) and the robotic arm (250). This facilitates removal and mounting of instruments (260) from the robotic arm (250) while the intervening adapter (410) remains in place. The intervening adapter (410) may be mounted prior to the beginning of the procedure.

In the configuration shown in FIGS. 4A and 4B, with an intervening adapter (410) being present, the adapter drive couplings (420) of the intervening adapter (410) may be directly mechanically engaged with the instrument holder drive couplings (416), once the intervening adapter (410) is installed on the instrument holder (214). Accordingly, rotation of an instrument holder drive coupling (416) directly translates into rotation of the corresponding adapter drive coupling (420).

Thus, the intervening adapter (410) establishes not only a barrier between the instrument holder (214) and the transmission assembly (370), but also introduces an additional mechanical element (the adapter drive coupling 420)) between the instrument holder drive coupling (416) of the instrument holder (214) and the receive coupling (378) of the transmission assembly (370). In some embodiments, the combination of the instrument holder drive coupling (416), the adapter drive coupling (420), and the receive coupling (378), establish a coupling like an Oldham coupling to compensate for possible radial offsets between the instrument holder drive coupling (416) and the receive coupling (378). In some embodiments, the instrument holder drive coupling (416), the adapter drive coupling (420), and the receive coupling (378) do not establish a coupling that compensates for radial offsets. In some embodiments, elements that establish a coupling that compensates for radial offset may be located elsewhere, e.g., in the instrument holder (214).

In embodiments that include one or more intervening adapters mounted to the robotic arm (250), such as an intervening adapter (410), the drive interface (418) is provided by the intervening adapter(s) (e.g., the intervening adapter (410)). In such embodiments, the intervening adapter (410) may also provide features (e.g., recesses, clips, etc.) to releasably lock the transmission assembly (370) of the instrument (260) in position.

In embodiments that do not include an intervening adapter (410), the drive interface (418) with the drive couplings and features to releasably lock the transmission assembly (370) in position may be located at the instrument holder (214). In such embodiments, the instrument holder drive couplings (416) are the drive couplings that directly engage with the receive couplings (378).

In one or more embodiments, engaging an instrument (260) involves the combination elements (400) of the robotic arm (250) and the instrument (260). The instrument (260) to be used in conjunction with the robotic arm (250) may be positioned as shown in FIG. 2. In this configuration, the transmission assembly (370) is disposed on the instrument holder (214), with the intervening adapter (410) providing a barrier (in a surgical example, the intervening adapter (410) helping to provide a sterile barrier). An engagement procedure, e.g., as described below in reference to FIG. 6, may be performed to enable a transmission of mechanical energy from the drive couplings to the receive couplings for actuation of the instrument (260). The drive couplings and receive couplings, specific design features of the drive couplings and the receive couplings, and possible benefits are subsequently described in reference to FIGS. 5A-5H.

While the above description covers embodiments without intervening adapters and with one or more intervening adapters mounted on the instrument holder of the robotic arm, one or more intervening adapters may alternatively or additionally be mounted on the transmission assembly (370), without departing from the disclosure.

FIGS. 5A-5H show example drive couplings and receive couplings in accordance with embodiments of the disclosure. The following paragraphs describe design features of the various drive couplings and receive couplings, followed by a discussion of the application of these drive couplings and receive couplings for the purpose of transmitting mechanical energy, and a discussion of possible benefits.

Turning to FIG. 5A, four different pairings of a first coupling with four different types of second couplings are shown. The first coupling may be a drive coupling and the second couplings may be receive couplings, or vice versa.

In one embodiment, the first coupling (500) has a single first feature (502) disposed on the first coupling (500). The single first feature (502) may be a protrusion that protrudes from a planar circular surface of the first coupling. The protrusion may be elongated in a radial direction as illustrated in FIG. 5A or may have other shapes. For example, the protrusion may be a peg (e.g., with a circular cross-section). In some instances, the first coupling (500) has no other features for engaging with receive couplings disposed on the first coupling. In some instances, the first coupling (500) has one or more additional features disposed on the first coupling and that differ geometrically from the single first feature (502), for engaging with receive couplings.

In one embodiment, the second coupling (505, 510, 515, 520) has one or more second features (507, 512, 517, 522) disposed on the second coupling (505, 510, 515, 520). Each of the one or more second features is physically configured for mechanically engaging with the same first feature (e.g., first feature (502)). For example, if an embodiment comprises second feature “A” and second feature “B”, both features being physically configured for mechanically engaging with a same first feature “1”, then either second feature “A” or second feature “B” may mechanically engage with first feature “1”. When multiple engagements are performed over time, sometimes a mechanical engagement may occur between second feature “A” and same first feature “1”, and sometimes mechanical engagement may occur between second feature “B” and same first feature “1”. In one embodiment, each of the one or more second features is a pocket in a planar surface of the second coupling. Each of the pockets may be shaped to accommodate the protrusion. The protrusion and the pocket(s) may be shape-matched such that mechanical backlash between the first and the second couplings, when mechanically engaged, is limited.

The first feature (502) of the first coupling (500) may mechanically engage with a second feature (507, 512, 517, 522) of the second coupling (505, 510, 515, 520). Depending on the configuration of the second coupling, the mechanical engagement may occur in different manners, as further discussed below with specific examples shown.

The second coupling (505) is equipped with a single second feature (507). Accordingly, one mechanical engagement orientation of the second coupling (505) relative to the first coupling (500) exists for the mechanical engagement.

The second coupling (510) is equipped with two second features (512). Accordingly, two mechanical engagement orientations exist for the mechanical engagement. The two mechanical engagement orientations have an offset of 180°.

The second coupling (515) is equipped with three second features. Accordingly, three mechanical engagement orientations exist for the mechanical engagement. The three mechanical engagement orientations have an offset of 120°.

The second coupling (520) is equipped with four second features. Accordingly, four mechanical engagement orientations exist for the mechanical engagement. The four mechanical engagement orientations have an offset of 90°.

Turning to FIG. 5B, two different pairings of a first coupling with two different types of second couplings are shown. The first coupling may be a drive coupling and the second couplings may be receive couplings or vice versa.

In one embodiment, the first coupling (530) has two first features (532) with an angular spacing of 180°. The two features (532) may be protrusions that protrude from a planar circular surface of the first coupling. The protrusions may be elongated in a radial direction as illustrated in FIG. 5B or may have other shapes.

In one embodiment, the second coupling (535, 540) has second features (537, 542) with an angular spacing of 180° and 90°, respectively. Each of the second features may be a pocket in a planar surface of the second coupling. Each of the pockets may be shaped to accommodate a protrusion. The protrusions and the pockets may be shape-matched such that mechanical backlash between the first and the second couplings, when mechanically engaged, is limited.

The first features (532) of the first coupling (530) may mechanically engage with second features (537, 542) of the second coupling (535, 540). Depending on the configuration of the second coupling, the mechanical engagement may occur in different manners, as subsequently discussed.

The second coupling (535) is equipped with two second features (537).

Accordingly, two mechanical engagement orientations of the second coupling (535) relative to the first coupling (530) exist for the mechanical engagement. The two mechanical engagement orientations have an offset of 180°.

The second coupling (540) is equipped with four second features (542). Accordingly, four mechanical engagement orientations of the second coupling (540) relative to the first coupling (530) exist for the mechanical engagement. The four mechanical engagement orientations have an offset of 90°.

Turning to FIGS. 5C1, 5C2, and 5C3, different versions of a pairing of a first coupling with a second coupling are shown. The first coupling may be a drive coupling and the second coupling may be a receive coupling or vice versa.

Referring to FIG. 5C1, in one embodiment, the first coupling (550) has three first features (552) with an even angular spacing of 120°. The three first features (552) may be protrusions that protrude from a planar circular surface of the first coupling. The protrusions may be elongated in a radial direction as illustrated in FIG. 5C1 or may have other shapes.

In one embodiment, the second coupling (555) has three second features (557) with an even angular spacing of 120°. Each of the second features may be a pocket in a planar surface of the second coupling. Each of the pockets may be shaped to accommodate a protrusion. The protrusions and the pocket may be shape-matched such that mechanical backlash between the first and the second couplings, when mechanically engaged, is limited.

The first features (552) of the first coupling (550) may mechanically engage with second features (557) of the second coupling (555). The second coupling (555) is equipped with three second features (557). Accordingly, three mechanical engagement orientations of the second coupling (555) relative to the first coupling (550) exist for the mechanical engagement. The three mechanical engagement orientations have an offset of 120°.

Referring to FIG. 5C2, in one embodiment, the first coupling (550) has a first set of three first features (552) with an even angular spacing of 120°, and a second set of three first features (553) with an even angular spacing of 120°. The three first features (552) in the first set and the three first features (553) in the second set may be protrusions that protrude from a planar circular surface of the first coupling. The protrusions may be elongated in a radial direction as illustrated in FIG. 5C2 or may have other shapes. The shapes of the protrusions in the first set are different from the shapes of the protrusions in the second set.

In one embodiment, the second coupling (555) has a first set of three second features (557) with an even angular spacing of 120°, and a second set of three second features (558) with an even angular spacing of 120°. Each of the second features in the first and second sets may be a pocket in a planar surface of the second coupling. Each of the pockets may be shaped to accommodate a protrusion.

The first features (552) in the first set of the first coupling (550) may mechanically engage with second features (557) in the first set of the second coupling (555). However, based on the geometry of the first and second features, the first features (552) in the first set of the first coupling (550) may not mechanically engage with second features (558) in the second set of the second coupling (555). In contrast, the first features (553) in the second set of the first coupling (550) may mechanically engage with second features (558) in the second set of the second coupling (555), whereas the first features (553) in the second set of the first coupling (550) may not mechanically engage with second features (557) in the first set of the second coupling (555). Analogous to the embodiment shown in FIG. 5C1, the embodiments of FIG. 5C2 provides three mechanical engagement orientations of the second coupling (555) relative to the first coupling (550). In presence of the additional features on the first and second couplings, higher torques may be transmitted.

The embodiment shown in FIG. 5C3 is similar to the embodiment shown in FIG. 5C2, in that it includes two different types of first and second features. The first coupling (550) has a single first feature (552) of a first type and a single first feature (553) of a second type. The second coupling (555) in FIG. 5C3, in the example, is identical to the second coupling (555) in FIG. 5C2.

Mechanical engagement of the first coupling (550) and the second coupling (555) in FIG. 5C3 may occur as described in reference to FIG. 5C2, with three possible engagement orientations. The number of possible engagement options may differ, without departing from the disclosure. For example, embodiments of the disclosure may have four or five engagement options.

Turning to FIG. 5D, a pairing of a first coupling with a second coupling is shown. The first coupling may be a drive coupling and the second coupling may be a receive coupling or vice versa.

In one embodiment, the first coupling (560) has four first features (562) with an even angular spacing of 90°. The four features (562) may be protrusions that protrude from a planar circular surface of the first coupling. The protrusions may be elongated in a radial direction as illustrated in FIG. 5D or may have other shapes.

In one embodiment, the second coupling (565) has four second features (567) with an even angular spacing of 90°. Each of the second features may be a pocket in a planar surface of the second coupling. Each of the pockets may be shaped to accommodate a protrusion. The protrusions and the pocket may be shape-matched such that mechanical backlash between the first and the second couplings, when mechanically engaged, is limited.

The first features (562) of the first coupling (560) may mechanically engage with the second features (567) of the second coupling (565). The second coupling (565) is equipped with four second features (567). Accordingly, four mechanical engagement orientations of the second coupling (565) relative to the first coupling (560) exist for the mechanical engagement. The four mechanical engagement orientations have an offset of 90°.

Turning to FIG. 5E, two different pairings of a first coupling with two different types of second couplings are shown. The first coupling may be a drive coupling and the second couplings may be receive couplings, or vice versa.

In one embodiment, the first coupling (570) has a first set of first features (572) and a second set of first features (574). In one embodiment, each of the first features in the first and second sets of features is a pocket in a planar surface of the first coupling. In the example as shown, the first set of first features (572) and the second set of first features (572) each include two features. A set of features may include any number of features, e.g., one, two, three, etc. features, without departing from the disclosure. In some embodiments, the features in the first set of features (572) have a first engagement geometry, and the features in the second set of features (574) have a second engagement geometry. The shape of the first engagement geometry is different from the shape of the second engagement geometry. In the example, it is the length of the pockets and the length of the protrusions that distinguish the first and second engagement geometries from each other. In another example, it is the position of the pockets and/or the position of the protrusion (e.g., in a radial direction from center for a disc, or a distance from a center or edge of the drive or receive coupling for a linear coupling) that distinguish the first and second engagement geometries from each other. In some examples, both shape and position distinguish the first and second engagement geometries from each other. Other characteristic dimensions may differ (e.g., one characteristic dimension being greater than another characteristic dimension, being located at different distances from the center of a coupling, etc.), without departing from the disclosure. The first and second engagement geometries may also have other distinguishing geometric features, without departing from the disclosure. In one embodiment, the second coupling (575, 580) has second features (577, 582) disposed on the second coupling (575, 580). In one embodiment, the second features (577, 582) are protrusions that protrude from planar surface of the second coupling (575, 580). The second features (577, 582) are physically configured for mechanically engaging with the first set of first features (572) and/or the second set of first features (574) of the first coupling (570). More specifically, the second features (577) have a first engagement geometry that corresponds to the second engagement geometry of the second set of first features (574), and the second features (582) have a second engagement geometry that corresponds to the first engagement geometry of the first set of first features (572). While the first engagement geometry of the second features (577) is not mechanically compatible with the first engagement geometry of the first set of first features (572), thereby preventing engagement of the first features (577) with the first set of first features (572), the second engagement geometry of the second features (582) is mechanically compatible with both the first engagement geometry of the first set of first features (572) and the first engagement geometry of the second set of first features (574). Accordingly, for a pairing of the second coupling (580) with the first coupling (570) four mechanical engagement orientations exist. The four mechanical engagement orientations have an offset of 90°. In contrast, for a pairing of the second coupling (575) with the first coupling (570) only two mechanical engagement orientations exist. The two mechanical engagement orientations have an offset of 180°. A mechanical coding is thus established, where different pairings of first and second couplings have different numbers of available mechanical engagement orientations. Additional examples of possible engagement geometries that may be used to establish mechanical coding are shown in FIGS. 5F and 5G. FIG. 5F shows a second coupling (584) with second features (585), and FIG. 5G shows a second coupling (586) with second features (587). While not shown, the corresponding first couplings may have first features with matching engagement geometries and/or non-matching engagement geometries. In some instances, mechanical tolerance is included in the design of these engagement geometries to allow for a radial offset compensation, as previously described.

The couplings discussed in reference to FIGS. 5A-5G comprise rotary disks that may be used to transmit rotational movements and torques. In contrast, 5H shows an example of an embodiment that is based on a linear slider that may be used to transmit translational movements and forces. In the example, a first coupling (590) has six first features (592). The first features (592) may be pockets in a planar surface of the first coupling (590). The second coupling (595) has a second feature (597). The second feature (597) may be a protrusion that protrudes from a planar surface of the second coupling (595). Each of the one or more first features is physically configured for mechanically engaging with the second feature. The first coupling may be a drive coupling and the second coupling may be a receive coupling, or vice versa. In the example, the first coupling (590) is equipped with six first features (592). Accordingly, six mechanical engagement positions exist for the mechanical engagement between the first coupling (590) and the second coupling (595). The translational offset between the engagement positions is determined by the spacing of the first features (592).

While FIGS. 5A-5H show various configurations of first couplings and second couplings, the disclosure is not limited to these configurations. For example, first couplings and second couplings may have any number of features, and the geometry of the features may deviate from what is shown in FIGS. 5A-5H, without departing from the disclosure. Specifically referring to the configuration of protrusions and pockets, protrusions may be in the form of pegs. These pegs may be spaced differently in a radial direction, yet a pocket may be sufficiently long (in a radial direction) to accommodate these differently spaced pegs. Further, the first couplings as described may serve as drive couplings or receive couplings with drive features and receive features, respectively. Similarly, the second couplings may serve as drive couplings and receive couplings with drive features and receive features, respectively.

The pairings of first couplings and second couplings as shown in FIGS. 5A-5G may be used to transmit mechanical energy (e.g., a torque and/or a velocity). In order to accurately transmit mechanical energy via a pairing of a first coupling and a second coupling, the one or more features of the first coupling are mechanically engaged with the one or more features of the second coupling. However, before an instrument interface is engaged with a drive interface, the receive coupling(s) of the instrument interface and their corresponding drive coupling(s) may be arbitrarily oriented relative to each other. Accordingly, when the engagement process first begins, the orientation of the drive feature(s) of the drive coupling and the receive feature(s) of the receive coupling are unlikely to allow mechanical engagement without relative motion of the receive features and the drive features. In one or more embodiments, the method for engaging the instrument, discussed below in reference to FIG. 6, involves a controlled rotation of the drive coupling, by the corresponding actuator. As the orientation of the drive coupling changes, eventually the orientation of the drive feature(s) matches the orientation of the receive feature(s) such that mechanical engagement between the drive feature(s) and the receive feature(s) can occur. In some instances, such mechanical engagements are facilitated by biasing forces (e.g. spring forces) pushing the drive coupling toward the receive coupling, and/or the receive coupling toward the drive coupling.

Depending on the initial orientation of the drive coupling and the receive coupling when physically coupling the receive interface of the instrument to the drive interface of the computer-assisted system, the drive coupling may need to cover a significant angular range until alignment between the feature(s) of the drive coupling and the feature(s) of the receive coupling is achieved. For example, referring to the first pairing in FIG. 5A, an angular range of up to +/−180° or 360° may need to be covered until alignment between the feature(s) of the drive coupling and the feature(s) of the receive coupling is achieved. Now referring to the second pairing in FIG. 5A, the angular range that may need to be covered is reduced to +/−90° or 180° because the second pairing provides two mechanical engagement orientations. Further, now referring to the third pairing in FIG. 5A, the angular range that may need to be covered is reduced to +/−60° or 120° because the third pairing provides three mechanical engagement orientations. Finally, now referring to the fourth pairing in FIG. 5A, the angular range that may need to be covered is reduced to +/−45° or 90° because the fourth pairing provides four mechanical engagement orientations.

The reduction of the angular range that may need to be covered until alignment between the feature(s) of the drive coupling and the feature(s) of the receive coupling is achieved enables an accelerated engagement of the instrument. The amount of time that is saved may be significant because the drive coupling, when covering the angular range may rotate at a limited angular velocity. The limited angular velocity may be necessary because, at higher velocities, the drive feature(s) and the receive feature(s) may fail to engage, as a result of tight mechanical tolerances between the drive feature(s) and the receive feature(s) intended to avoid mechanical backlash.

Now again referring to FIG. 5A, the time needed for engagement when using the second pairing may be reduced by 50%, compared to the first pairing. The time needed for engagement when using the third pairing may be reduced by 67%, compared to the first pairing. The time needed for engagement when using the fourth pairing may be reduced by 75%, compared to the first pairing. Accordingly, the availability of more engagement orientations results in a reduction of the time needed to complete the engagement. However, for reasons discussed in reference to FIG. 6, the number of engagement orientations that may be used may be limited by other factors that may be specific to an instrument and/or individual controllable degrees of freedom of the instrument. Accordingly, it may be desirable to customize the availability of engagement orientations in an instrument-specific and/or degree of freedom-specific manner.

Instrument-specific customization may be accomplished, for example, by using a drive coupling that is compatible with multiple different types of receive couplings. Consider a scenario that involves the use of an instrument A suitable for engagement at two engagement orientations and the use of an instrument B suitable for engagement at four engagement orientations. In this scenario, a drive coupling based on the first coupling (500) of FIG. 5A may be used to engage with a receive coupling based on the second coupling (510) of FIG. 5A for instrument A. Further, for instrument B, the first coupling (500) may be used to engage with a receive coupling based on the second coupling (520) of FIG. 5A. Alternatively, the configurations shown in FIG. 5B may be used.

Degree of freedom-specific customization may be accomplished in a similar manner. For example, now referring to FIGS. 3, 4A, and 4B, all five drive couplings (420) may be of the same type. Assume, for example, that all five drive couplings are based on the first coupling (500) of FIG. 5A. In order to accomplish the degree of freedom-specific customization, the five receive couplings (378) may be of different types as needed.

While FIGS. 1, 2, 3, 4A, 4B, and 5A-5H show various configurations of components, other configurations may be used without departing from the scope of the disclosure. For example, various components may be combined to create a single component. As another example, the functionality performed by a single component may be performed by two or more components. While a particular instrument with a particular end effector and particular degrees of freedom is shown in FIG. 3, the disclosure generalizes to any type of instrument with any type and number of degrees of freedom. Also, while the instrument is described as being supported by a robotic arm with an instrument holder, the instrument may be supported by any type of repositionable structure, without departing from the disclosure. Further, while most of the discussion of the use and design referred to rotary disk-shaped couplings, e.g., as shown in FIGS. 5A-5G, the discussion is equally applicable to linear slider-shaped couplings, e.g., as shown in FIG. 5H.

Turning to FIG. 6, a flowchart in accordance with one or more embodiments is shown. The flowchart of FIG. 6 depicts a method (600) for computer-assisted systems. The method (600) may be used to engage an instrument of a computer-assisted system. One or more of the steps in FIG. 6 may be performed by various components of systems, previously described with reference to FIGS. 1, 2, 3, 4A, 4B, and 5A-5H. The method may be executed on one or more processors, e.g., of the control system of the computer-assisted system. While the flowcharts are for a system that uses rotary disk-shaped drive couplings and receive couplings, a similar method may be used for linear slider-shaped drive and receive couplings, without departing from the disclosure.

While the various steps in the flowchart are presented and described sequentially, one of ordinary skill will appreciate that some or all of the steps may be executed in different orders, may be combined or omitted, and some or all of the steps may be executed in parallel. Additional steps may further be performed. Furthermore, the steps may be performed actively or passively. For example, some steps may be performed using polling or be interrupt driven in accordance with one or more embodiments of the invention.

Turning to the flowchart, in Step 602, a physical coupling of the receive interface of the instrument with the drive interface of the computer-assisted system is detected. The physical coupling may be detected in various manners. In one example, a contact or a switch may be activated to signal the physical coupling. The contact or switch may activate when the instrument is releasably locked onto, for example, an instrument holder of the computer-assisted system, as previously described. Alternatively, a user input may confirm the physical coupling.

Step 604 illustrates that optional steps may be added to method 600. Instances that do not include Step 604 would proceed directly from Step 602 to Step 606. In optional Step 604, in response to detecting the physical coupling, it is determined whether the receive couplings of the instrument have multiple engagement options with the drive couplings of the drive interface. Different instruments may be configured differently. For example, an instrument A may have a receive coupling with a single engagement option, whereas an instrument B may have a receive coupling with four engagement options. Also, different receive couplings of a single instrument may have different numbers of engagement options. The detection of the multiple engagement options may involve a lookup operation in an instrument database, once the instrument has been identified. The instrument may be identified, for example based on an instrument coding, e.g., by an electronic chip of the instrument, a mechanical coding, or based on a user input. In instances that include Step 604, after completion of Step 604, the number of engagement options of each of the receive couplings of the instrument are known.

In Step 606, a motion of the drive coupling is caused to engage the drive coupling with the receive coupling. The motion of the drive coupling may be selected to be sufficient to enable the engagement in only one engagement option of the plurality of engagement options. Consider, for example, a receive coupling with four rotary engagement options. The four engagement options, in the example, are equally separated by a 90° angle. In the example, the motion of the drive coupling may be a rotation of the drive coupling. The rotation may be limited to an angle that is sufficient to achieve engagement for one of the engagement options. For example, the rotation may be set to an amount close to 90 degrees, be limited to 95°, etc. In some instances, the rotation may be set to an amount, or be limited to an amount, that provides multiple engagement possibilities. In the above example, such a rotation may be set to close to 180°, be limited to 185°, etc. to provide two engagement possibilities. Additional examples include rotations that provide three, four, five or more engagement possibilities. In the above example, motion of the drive coupling may comprise rotations of 270, 360, 450, or more degrees, or be limited to 275, 365, 455, or more degrees. In linear examples, the number of engagement options in which the drive and receive couplings may engage may be defined by an amount of translation caused of the drive coupling.

The motion of the drive coupling may be performed by commanding a first speed of the drive coupling. An actuator associated with the drive coupling may drive the first drive coupling at the first speed, based on a command to cause engagement. The first speed may be set at a value, or limited by a speed threshold to a value, more likely to allow for engagement, given the physical geometries and materials of the drive and receive couplings. In many instances, the likeliness of the receive features and the drive feature(s) failing to engage are higher at higher speeds that provide very short durations in which the receive and drive features may engage.

In one embodiment, a successful engagement is detectable by a sensor (e.g., an engagement sensor). Such an engagement sensor may be dedicated to sensing engagement, or may sense some other parameter (e.g., electrical communications between the robotic arm and the instrument) indicative of engagement or the lack of engagement. In such a case, the first motion may be performed until the successful engagement is detected. The sensor may sense a resistance encountered as a result of the actuator beginning to drive the transmission elements associated with the receive coupling, after engagement. The sensor may measure, for example, a motor torque or current. The sensor may also directly sense the engagement, e.g., as the drive features engage with corresponding receive features, which may result in a measurable position change, e.g., of the drive coupling.

The second motion may be selected to be sufficient to enable the engagement in the one engagement option. For example, the rotation may be 365° rotation. The second motion may otherwise be similar to the first motion.

For an instrument with multiple receive couplings, the operations of Step 606 may be performed either sequentially or in parallel, for the multiple receive couplings. While an engagement could be accomplished by universally performing the second motion, use of the second motion where possible reduces the time needed until engagement is accomplished.

In Step 608, it is confirmed whether the drive coupling has engaged with the receive coupling. In one or more embodiments, the confirmation comprises causing driving of the drive coupling by the corresponding actuator, and determining if a mechanical stop is encountered by the instrument during the driving. Encountering a mechanical stop may serve as an indication for successful engagement, because the mechanical stop is reached only if the drive coupling is engaged with the receive coupling to cause movement on the instrument side. No mechanical stop would be reached if the engagement of the drive coupling with the receiving coupling had been unsuccessful. A mechanical stop may be caused by a mechanical limit of the instrument (e.g., a hard stop) or by an obstacle encountered during the driving. The detection of the mechanical stop may be performed at the actuator that drives the drive coupling, e.g., by an encoder, resolver, or any other type of position or velocity sensor that is capable of detecting the blockage of further movement when the mechanical stop is reached.

The movement towards the mechanical stop may be performed at a second speed of the drive coupling. The second speed of the drive coupling may be significantly higher than the first speed of the drive coupling (used in Step 606). However, the second speed may be limited by mechanical considerations such as inertia. More specifically, the inertia of the actuator may result in a significant torque peak when the mechanical stop is abruptly reached. The second speed may, thus, be selected not to exceed a value that avoids mechanical overloading when abruptly encountering the mechanical stop. The torque peak may depend on the gear ratio of the transmission elements and further on whether the transmission elements are stiff or elastic. For example, in the instrument (260) described in reference to FIG. 3, a high gear reduction ratio is used for rolling the instrument along the roll axis. The gear reduction is accomplished using spur gears, i.e., transmission elements that are stiff. Accordingly, inertia on the actuator side has the potential to produce a high torque peak when the mechanical stop is reached and limits the second speed to avoid damage to the transmission elements. In contrast, other transmission elements may have a lesser gear reduction ratio and may be somewhat elastic (e.g., as a result of using tensions members), and the corresponding drive couplings may, thus, be driven at a higher second speed.

If a mechanical stop is not reached, it may be concluded that the previously performed engagement has failed. Another attempt may be performed and/or an error message may be provided.

In Step 610 the engagement option of the plurality of engagement options (engagement orientation for rotary drive couplings, engagement position for linear drive couplings) in which the drive coupling and the receive coupling are engaged is identified. The identification may be performed based on encountering the mechanical stop as subsequently discussed.

Regardless of whether there is a single engagement option or multiple engagement options, there may be ambiguity between the orientation on the actuator side and the orientation on the instrument side. More specifically, the engaged drive coupling and receive coupling may complete multiple turns between two hard stops that delimit the maximum range of motion, and the engagement may have occurred anywhere between these two hard stops. Accordingly, no unambiguous mapping between orientation on the actuator side and orientation on the instrument side is initially available. In other words, orientations on the instrument side cannot be predicted based on known orientations on the actuator side.

In one or more embodiments, to resolve this ambiguity, information about known hard stops and kinematics of the instrument are used to enable a mapping from orientation on the actuator side to orientation on the instrument side. The relationship between orientations on the actuator side and orientations on the instrument side may be parameterized as follows:

$${θ}_{instrument} = C ( {θ}_{disc} - {θ}_{offset} + 2 pi n ) ,$$

θᵢₙₛₜᵣᵤₘₑₙₜ is the vector of orientations on the instrument side (in the example of the instrument of FIG. 3, the vector includes five elements such as the orientation of the end effector in four degrees of freedom and the rotation of the instrument shaft);
C is a coupling matrix that relates the vector of orientations of the receive couplings to the vector of instrument orientations, e.g., to fully describe the kinematic configuration of the instrument for any combination of orientations of the receive couplings. The coupling matrix may include interaction terms that reflect kinematic interactions between the degrees of freedom of the instrument (in the example of the instrument of FIG. 3, the C is a 5×5 matrix);
θ_{disc} is the vector of orientations of the drive couplings (in the example of the instrument of FIG. 3, the vector has five elements);
θ_{offset} is the vector of offsets as measured during manufacturing (in the example of the instrument of FIG. 3, the vector has five elements and captures the offsets to the receive couplings when the instrument is in a particular configuration, e.g., when the wrist is straight); and n is a vector of values which is chosen based on finding the hard stop and is to be identified (in the example of the instrument of FIG. 3, the vector has five elements). The selection of the values in n is subsequently described.

n may be calculated as follows. Based on reaching the mechanical stops when executing Steps 608 and or 610, it is assumed that the mechanical stops are the hard stops of the instrument. The joint orientations at the hard stops are known (known instrument parameter). Further, θ_{disc} is known from the encoder readings of the actuators, and θ_{offset} is known (known instrument parameters). Accordingly, the above equation may be solved for the only unknown, n.

The solution may not result in integer values due to inaccuracies such as inaccurate knowledge about the hard stops, elasticities in the transmission elements, etc. Accordingly, each element of n may be rounded. Rounding may be performed to the nearest integer in case of a single engagement option. To accommodate receive couplings with multiple engagement options, the rounding may be performed to the nearest half-integer (for two engagement options, quarter-integer (for four engagement options), etc. Accordingly, the values in n may be integers, half-integers, quarter-integers, etc.

One example for variability when relying on a hard stop is specific to cable-driven joints. In the example, the hard stop may be based on the tip of the end effector pressing against an internal wall of the cannula (when the end effector of the instrument is inside the cannula), in this mechanical configuration, there may be some variability in the location of the hard stop. Further, cables have flexibility and are subject to stretching over life which may alter the relationship between the hard stop encountered at the end effector and the corresponding orientations of the drive couplings. These factors may contribute to some degree of uncertainty in the location of the hard stop. This uncertainty may limit the number of engagement options, because additional engagement options can be made available only to the extent that the operations of Step 614 enable their disambiguation in presence of the described uncertainties. In absence of flexibility or elasticity, the described operations may be used to discriminate between a higher number of engagement options. Accordingly, in the example of the instrument of FIG. 3, the gear-driven rolling of the instrument axis may be a particularly good candidate for a higher number of engagement options.

In Step 612, with the drive coupling engaged with the receive coupling, the drive coupling is commanded to transmit, through the receive coupling and a transmission element coupled with the receive coupling, mechanical energy to a joint or end effector of the instrument. Step 612 may be performed simultaneously for all drive couplings and receive couplings, e.g., when performing a procedure with the instrument after completion of the instrument engagement. The execution of Step 612 is not mandatory. Specifically, Step 612 is executed only if mechanical energy is to be transmitted to a joint or end effector of the instrument. Step 612 may not be executed if the system is in an idle state (no transmission of mechanical energy to a joint or end effector).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A computer-assisted system comprising:
an instrument comprising:
a receive interface configured to couple with a drive interface, the receive interface comprising a first receive coupling with a plurality of first receive features disposed on the first receive coupling, each first receive feature of the plurality of first receive features physically configured for mechanically engaging with a same first drive feature disposed on a first drive coupling of the drive interface, and
a transmission element coupled with the first receive coupling such that mechanical energy transmitted by the first drive coupling to the first receive coupling is transmitted to the transmission element.

2. The computer-assisted system of claim 1, wherein each first receive feature of the plurality of first receive features is physically configured for mechanically engaging with a same second drive feature disposed on the first drive coupling.

3. The computer-assisted system of claim 1, wherein the plurality of first receive features comprises at least four first receive features.

4. The computer-assisted system of claim 1,
wherein the receive interface further comprises: a plurality of second receive features disposed on the first receive coupling, each second receive feature of the plurality of second receive features physically configured for mechanically engaging with a same second drive feature disposed on the first drive coupling and for not mechanically engaging with the same first drive feature;
wherein each of the first receive features has a first engagement geometry; and
wherein each of the second receive features has a second engagement geometry different from the first engagement geometry.

5. The computer-assisted system of claim 4, wherein the first engagement geometry differs from the second engagement geometry by:
the first and second engagement geometries having different shapes; or
the first and second engagement geometries being located at different distances from a center of the receive coupling.

6. The computer-assisted system of claim 1, further comprising a robotic arm, the robotic arm comprising the drive interface,
wherein the receive interface further comprises: a plurality of second receive features disposed on the first receive coupling, each second receive feature of the plurality of second receive features physically configured for mechanically engaging with a same second drive feature disposed on the first drive coupling and for not mechanically engaging with the same first drive feature;
wherein the same first drive feature has a first engagement geometry;
wherein the same second drive feature has a second engagement geometry; and
wherein a shape of the second engagement geometry is different from a shape of the first engagement geometry.

7. The computer-assisted system of claim 1, further comprising:
a robotic arm comprising the drive interface, wherein
more receive features are disposed on the first receive coupling than drive features are disposed on the first drive coupling.

8. The computer-assisted system of claim 1, wherein:
the instrument comprises an intervening adapter, and the intervening adapter provides the receive interface; or
the computer-assisted system comprises an intervening adapter, and the intervening adapter provides the drive interface.

9. The computer-assisted system of claim 1,
wherein each receive feature of the plurality of receive features comprises a pocket in the first receive coupling; and
wherein the first drive feature comprises a protrusion from the first drive coupling.

10. The computer-assisted system of claim 1,
wherein the first receive coupling comprises a rotary disk with receive features of the plurality of receive features disposed on the rotary disk; or
wherein the first receive coupling comprises a linear slider with receive features of the plurality of receive features disposed on the linear slider.

11. The computer-assisted system of claim 1,
wherein the receive interface further comprises a second receive coupling with a plurality of second receive features disposed on the second receive coupling, each second receive feature of the plurality of second receive features physically configured for mechanically engaging with a same second drive feature disposed on a second drive coupling of the drive interface; and
wherein a count of the first receive features disposed on the first receive coupling is different from a count of the second receive features disposed on the second receive coupling.

12. The computer-assisted system of claim 1, further comprising:
a robotic arm comprising the drive interface; and
a control system comprising one or more processors, the control system configured to:
detect a physical coupling of the receive interface of the instrument with the drive interface of the computer-assisted system,
cause, with the control system and in response to detecting the physical coupling, motion of the first drive coupling to engage the first drive coupling with the first receive coupling; and
confirming, with the control system, whether the first drive coupling has engaged with the first receive coupling.

13. The computer-assisted system of claim 12, wherein the control system is further configured to:
determine, in response to detecting the physical coupling, whether the first receive coupling has multiple engagement options with the first drive coupling,
wherein causing the motion of the first drive coupling comprises:
causing a first motion in response to determining that the first receive coupling has a plurality of engagement options with the first drive coupling, and
causing a second motion in response to determining that the first receive coupling does not have a plurality of engagement options with the first drive coupling.

14. The computer-assisted system of claim 13,
wherein determining whether the first receive coupling has multiple engagement options with the first drive coupling comprises:
determining a type of the instrument.

15. The computer-assisted system of claim 12,
wherein confirming whether the first drive coupling has engaged with the first receive coupling comprises:
causing, by the control system, driving of the first drive coupling; and
determining, by the control system, if a mechanical stop is encountered by the instrument during the driving.

16. The computer-assisted system of claim 15, wherein the receive coupling has a plurality of engagement options with the drive coupling, and wherein confirming whether the first drive coupling has engaged with the first receive coupling further comprises:
identifying, based on encountering the mechanical stop, in which engagement option of the plurality of engagement options that the drive and receive couplings have engaged.

17. The computer-assisted system of claim 16, wherein identifying the engagement option in which the drive coupling and the receive coupling are engaged comprises:
identifying which first receive feature of the plurality of first receive features has engaged with the same first drive feature; or
sensing an orientation of the drive coupling when the mechanical stop is encountered, and establishing a mapping between the orientation of the drive coupling and a known kinematic configuration of the instrument at the mechanical stop.

18. The computer-assisted system of claim 12, wherein causing the motion of the first drive coupling comprises:
commanding a first speed of the first drive coupling at a first time; and
commanding a second speed of the first drive coupling at a second time later than the first time,
wherein the second speed is higher than the first speed.

19. The computer-assisted system of claim 12, wherein the control system is configured to confirm whether the first drive coupling has engaged with the first receive coupling by:
determining if a resistance associated with the first drive coupling during the causing the motion of the first drive coupling corresponds to engagement; or
determining if an engagement sensor provides a signal indicating that the engagement has occurred.

20. A method for engaging an instrument of a computer-assisted system, comprising:
detecting, with a control system of the computer-assisted system, a physical coupling of a receive interface of the instrument with a drive interface of the computer-assisted system, wherein the receive interface comprises a receive coupling and the drive interface comprises a drive coupling;
causing, with the control system and in response to detecting the physical coupling, motion of the drive coupling to engage the drive coupling with the receive coupling, wherein the receive coupling has a plurality of engagement options with the drive coupling; and
confirming, with the control system, whether the drive coupling has engaged with the receive coupling.

21. The method of claim 20,
wherein a plurality of first receive features is disposed on the receive coupling; and
wherein identifying the engagement option in which the drive coupling and the receive coupling are engaged comprises:
identifying which first receive feature of the plurality of first receive features has engaged with a same drive feature of the drive coupling.

22. The method of claim 20, wherein causing the motion of the drive coupling comprises:
determining, with the control system and in response to detecting the physical coupling, whether the receive coupling has multiple engagement options with the drive coupling;
causing a first motion in response to determining that the receive coupling has multiple engagement options with the drive coupling; and
causing a second motion in response to determining that the receive coupling does not have multiple engagement options with the drive coupling.

23. The method of claim 22, wherein determining whether the receive coupling has multiple engagement options with the drive coupling comprises:
determining a type of the instrument.

24. The method of claim 20, wherein confirming whether the drive coupling has engaged with the receive coupling comprises:
causing, by the control system, driving of the drive coupling; and
determining, by the control system, if a mechanical stop is encountered by the instrument during the driving.

25. The method of claim 24, wherein confirming whether the drive coupling has engaged with the receive coupling further comprises:
identifying, based on encountering the mechanical stop, an engagement option of the plurality of engagement options in which the drive coupling and the receive coupling are engaged.

26. The method of claim 20, wherein causing the motion of the drive coupling comprises:
commanding a first speed of the drive coupling at a first time; and
commanding a second speed of the drive coupling at a second time later than the first time,
wherein the second speed is higher than the first speed.

27. A non-transitory machine-readable medium comprising a plurality of machine-readable instructions executed by one or more processors associated with a computer-assisted system, the plurality of machine-readable instructions causing the one or more processors to perform a method for engaging an instrument, the method comprising:
detecting, with a control system of the computer-assisted system, a physical coupling of a receive interface of the instrument with a drive interface of the computer-assisted system, wherein the receive interface comprises a receive coupling and the drive interface comprises a drive coupling;
causing, with the control system and in response to detecting the physical coupling, motion of the drive coupling to engage the drive coupling with the receive coupling, wherein the receive coupling has a plurality of engagement options with the drive coupling; and
confirming, with the control system, whether the drive coupling has engaged with the receive coupling.

28. The non-transitory machine-readable medium of claim 27, wherein causing the motion of the drive coupling comprises:
determining, in response to detecting the physical coupling, whether the receive coupling has multiple engagement options with the drive coupling;
causing a first motion in response to determining that the receive coupling has multiple engagement options with the drive coupling, and
causing a second motion in response to determining that the receive coupling does not have multiple engagement options with the drive coupling.

29. The non-transitory machine-readable medium of claim 27, wherein confirming whether the drive coupling has engaged with the receive coupling comprises:
causing driving of the drive coupling; and
determining if a mechanical stop is encountered by the instrument during the driving.
